(19) United States
(12) Patent Application Publication
(10) Pub. No.: US 2026/0356802 A1
(43) Pub. Date: Nov. 12, 2026

(54) ION EXCHANGE OF PYRIDINIUM OXIME SALTS

(71) Applicant: Southwest Research Institute, San Antonio, TX (US)

(72) Inventors: Shawn T. BLUMBERG, San Antonio, TX (US); Antonio A. MENCHACA, Seguin, TX (US); Dillon P. CAO, San Antonio, TX (US)

(21) Appl. No.: 19/180,341

(22) Filed: Apr. 16, 2025

(51) Int. Cl. C07D 213/38; C07D 213/30

(52) U.S. Cl. CPC C07D 213/38; C07D 213/30

(57) ABSTRACT

A method for ion exchange of pyridinium oxime salts In particular, formation of desired pyridinium oxime salts as antidotes to treat organophosphorus nerve agent exposure.

### FIELD

The present disclosure is directed at a method for ion exchange of pyridinium oxime salts. In particular, the formation of desired pyridinium oxime salts as antidotes to treat organophosphorus nerve agent exposure.

### BACKGROUND

Organophosphorus nerve agents (OPNA), used as chemical weapons and pesticides, irreversibly inhibit acetylcholinesterase (AChE) and cause an estimated 300,000 deaths per year worldwide. A number of pyridinium oxime antidotes are available to treat OPNA poisoning, including 1-[({2,4-bis[(E)-(hydroxyimino)methyl]-1-pyridiniumyl}methoxy)methyl]-4-carbamoylpyridinium dimethanesulfonate or HLö-7 DMS (1), 1,1′-methylenebis(4-((hydroxyimino)methyl)-pyridinium)dimethanesulfonate or MMB-4 DMS (2), (1-(2-(hydroxyiminomethyl)pyridinium)-3-(4-carbamoylpyridinium)-2-oxapropane)dimethanesulfonate or HI-6 DMS (3), pralidoxime chloride or 2-PAM CI (4), and 1,1′(oxydimethylenebis(4-formylpyridinium)dioxime dimethanesulfonate or obidoxime DMS (5). See FIG. 1 where (⁻OMs) is reference to ⁻OSO₂CH₃.

Relatively large doses are typically required to treat OPNA poisoning and, due to the speed at which nerve agents take effect, these antidotes must dissolve rapidly. A number of salt species have been investigated to fine-tune both the kinetic and thermodynamic solubilities of these antidotes, finding that either the chloride or methane sulfonate salts to be optimal. See again, FIG. 1. Yet due to the synthesis of many of these antidotes, iodide, bromide or other pseudo halogen salt is produced as an intermediate. This necessitates a final conversion to the desired salt-form in an additional step.

The final salt exchange is therefore usually mediated by an anion-exchange resin as exemplified in FIG. 2. The first step in a typical ion-exchange is preparation of the ion-exchange resin with the desired anion (8) shown here as Y⁻. A quaternary ammonium hydroxide resin (6) is purchased, of which there are many bulk suppliers. This basic resin is neutralized with the conjugate acid of the desired anion to form the desired anion exchange resin (8). This is typically done in water, and the resulting resin is washed and dried prior to use. The pyridinium oxime antidote (7) is then dissolved and passed through the resin to replace the undesired anion (X⁻) with the desired anion (Y⁻) in the resin to furnish 9, namely the pyridinium oxime with Y⁻. While this procedure is generally useful, there are several issues that make its utilization inconvenient on large scales: 1) around 10-20 g/g resin loading is typically required for quantitative anion conversion, which requires the API to be dissolved in large volumes of solvent; (2) the desired anion exchange resin takes an extra step to prepare, which adds cost to processes and then must be characterized as a current good manufacturing practice (cGMP) starting material if being used in the production of clinical batches; and (3) produces significant waste in the form of spent resin and solvent used in its preparation and use.

### SUMMARY

A method of exchanging anionic groups in a pyridinium oxime salt comprising:
a. providing a first pyridinium oxime salt having the following structure:

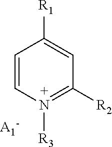

wherein A₁⁻ comprises a first anionic group, R₁ comprises a hydrogen or oxime group, R₂ comprises a hydrogen or oxime group and R₃ comprises one of:
(1) a terminal hydrocarbon group; or
(2) a hydrocarbon linking group R₄ connected to the nitrogen atom of a second pyridinium oxime as in the following:

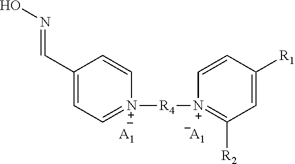

(3) a hydrocarbon ether linking group (—R₅—O—R₅—) where R₅ is a hydrocarbon connected to the nitrogen atom of a second heterocyclic ring nitrogen atom as in one of the following

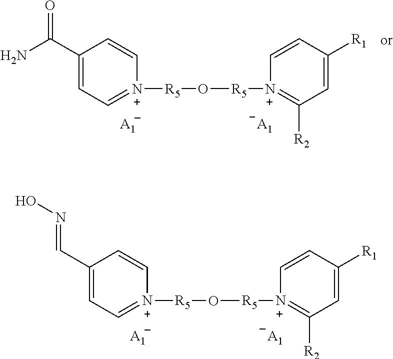

b. transforming said first pyridinium oxime salt into a zwitterionic intermediate and treating said zwitterionic intermediate with a proton acid HA₂ where His a hydrogen and A₂ forms a second anionic group A₂⁻ thereby providing a second pyridinium oxime salt having one of the following structures:

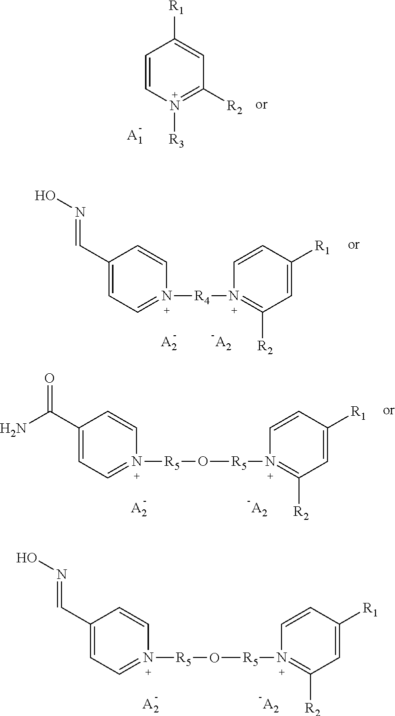

wherein A₂⁻ comprises an anionic group that is different from the first anionic group A₁⁻.

A method of exchanging anionic groups in a pyridinium oxime salt comprising:
a. providing a first pyridinium oxime salt in a polar solvent layer having the following structure:

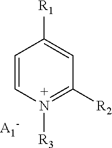

wherein A₁⁻ comprises a first anionic group, R₁ comprises a hydrogen or oxime group, R₂ comprises a hydrogen or oxime group and R₃ comprises one of:
(1) a terminal hydrocarbon group; or
(2) a hydrocarbon linking group R₄ connected to the nitrogen atom of a second pyridinium oxime as in the following:

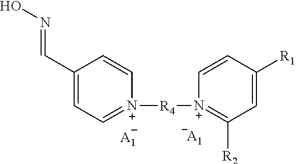

(3) a hydrocarbon ether linking group (—R₅—O—R₅—) where R₅ is a hydrocarbon connected to the nitrogen atom of a second heterocyclic ring nitrogen atom as in one of the following

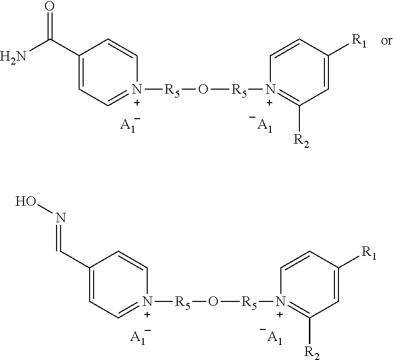

b. providing a tertiary amine X₃N in a non-polar solvent layer where X is a hydrocarbon wherein said non-polar solvent layer is in contact with said first pyridinium oxime salt in said polar solvent layer;
c. transforming said first pyridinium oxime salt in said polar solvent layer into a zwitterionic intermediate and separating said zwitterionic intermediate in said polar solvent layer from said non-polar solvent layer and treating said zwitterionic intermediate with a proton acid HA₂ where H is a hydrogen and A₂ forms a second anionic group A₂⁻ thereby providing a second pyridinium oxime salt having one of the following structures:

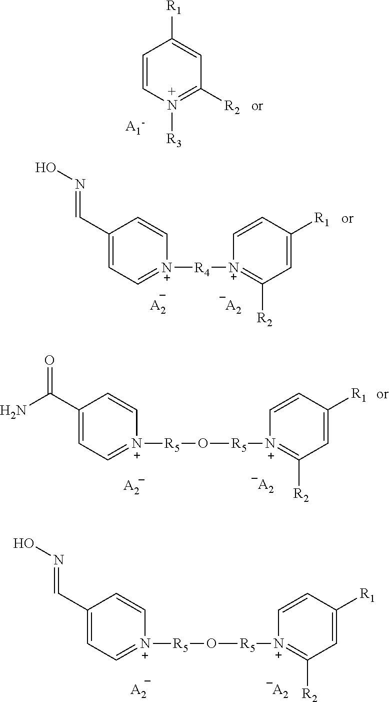

wherein A₂⁻ comprises an anionic group that is different from the first anionic group A₁⁻.

### BRIEF DESCRIPTION OF THE DRAWINGS

The above-mentioned and other features of this disclosure, and the manner of attaining them, will become more apparent and better understood by reference to the following description of embodiments described herein taken in conjunction with the accompanying drawings, wherein:

FIG. 1 illustrates the identified and reported pyridinium oxime antidotes to treat OPNA poisoning.

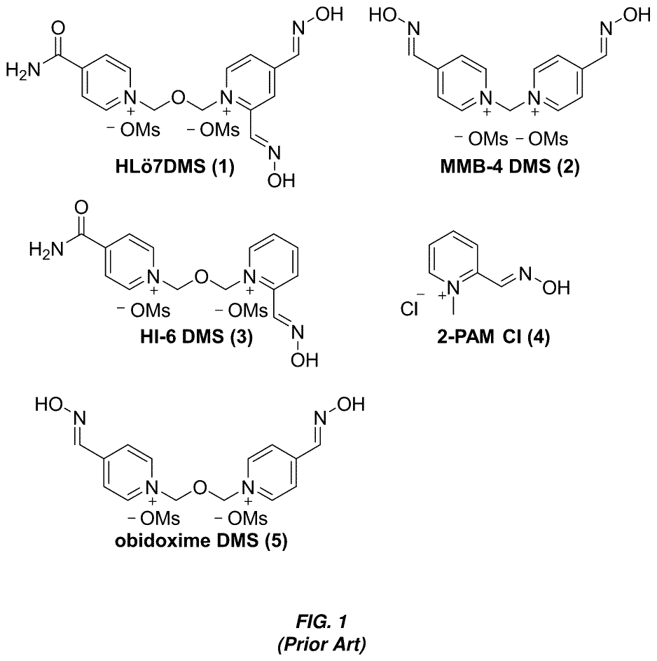

FIG. 2 illustrates the reported use of anion exchanges resins for the treatment of oxime antidotes.

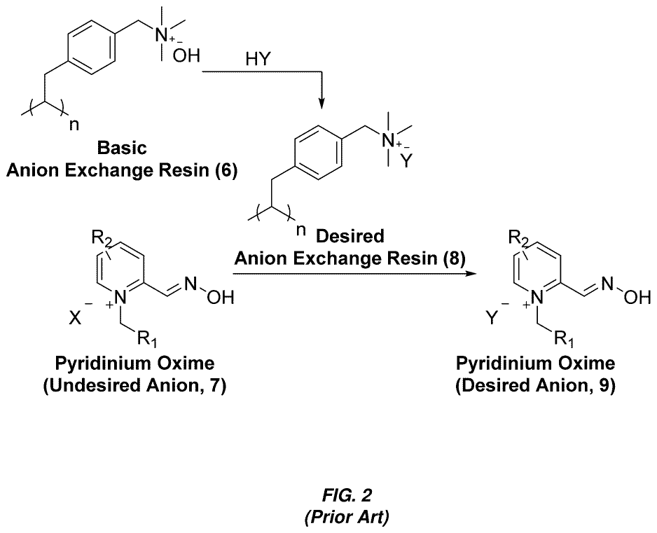

FIG. 3 illustrates a preferred procedure for the ion-exchange of a first cationic pyridinium oxime having an anion A₁⁻ for replacement where the tertiary amine is soluble in the non-polar solvent layer and the pyridine oxime and formed zwitterionic intermediate are formed in the polar solvent layer.

FIG. 4 illustrates the preferred recovery procedure for the zwitterionic intermediate formed in FIG. 3.

### DETAILED DESCRIPTION OF THE PREFERRED EMBODIMENTS

The present invention provides for ion-exchange of a first cationic pyridinium oxime (first pyridinium oxime salt) having an anion for replacement via the formation and use of zwitterionic intermediates. The zwitterionic intermediates are then preferably recovered by phase separation followed by conversion into a second cationic pyridinium oxime (second pyridinium oxime salt) having the desired anion. The process is therefore one that can avoid the use of anion exchange resin treatment of pyridinium oxime salts.

As an initial matter, the pyridine oxime salts that are suitable for ion-exchange from a first cationic pyridinium oxime having a first anion for replacement to a second cationic pyridinium oxime salt having a desired anion, preferably include those oximes that are suitable to react with AChE-nerve agent adduct, to cleave the nerve agent into harmless and metabolized fragments as well as restore the catalytic AChE activity. The first pyridinium oxime salt may therefore be represented as having the following moiety within the overall pyridinium oxime antidote:

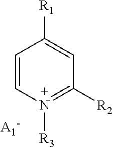

In the above, A₁⁻ represents the first anion that is to be replaced and which may therefore include iodide (I⁻), bromide (Br⁻) and what may be termed pseudo halogens that form univalent anions with a pyridinium oxime. A pseudo halogen may therefore be understood as any anion whose pKa is greater or equal to HCL, that is not Br or I. R₁ comprises a hydrogen or oxime group, R₂ comprises a hydrogen or oxime group and R₃ comprises one of: (1) a terminal hydrocarbon group such as a an alkyl group, e.g., a —CH₃ group; or (2) a hydrocarbon linking group R₄ such as a methylene group (—CH₂—) connected to the nitrogen atom of a second pyridinium oxime as in the following:

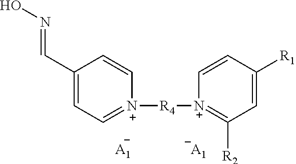

or
(3) a hydrocarbon ether linking group (—R₅—O—R₅—) where R₅ is a hydrocarbon such as a dimethylene ether group ((—CH₂—O—CH₂—) connected to the nitrogen atom of a second heterocyclic ring nitrogen atom as in one of the following

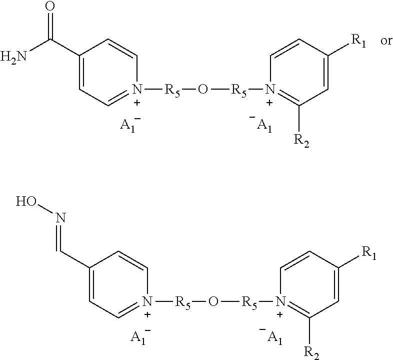

The first pyridinium oxime salt moiety noted above is then transformed into a zwitterionic intermediate (i.e. a compound having both positive and negative charge yet having an overall neutral charge). Preferably, this can be achieved by treatment with a tertiary amine (X₃N) where X comprises a hydrocarbon group. The hydrocarbon group preferably comprises an alkyl group, an unsaturated alkyl group, or an aromatic group. As shown below, in the case where R₁ is a hydrogen and R₂ is an oxime group, the first pyridinium oxime salt containing the undesired anion A₁⁻, upon treatment with the tertiary amine X₃N can be converted into a zwitterionic intermediate, which may also be identified as a zwitterionic base form:

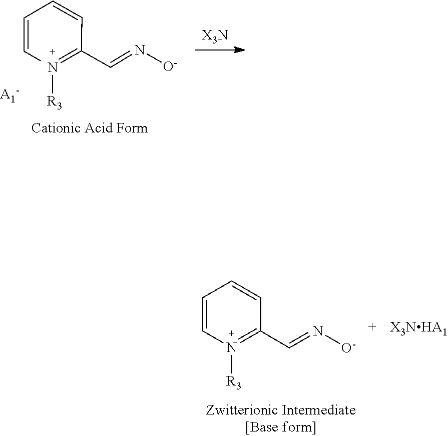

The zwitterionic intermediate is then treated with an appropriate proton acid (e.g. HA₂) where H is hydrogen and A₂ provides the anion A₂⁻ to be introduced into the pyridinium oxime salt to replace the first anionic group A₁⁻, as illustrated below:

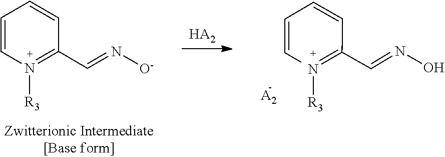

In the above, HA₂ is an acid containing the desired moiety (A₂) that is to then be introduced as an anionic moiety (A₂⁻) to the zwitterionic intermediate. As shown, this results in the formation of a second pyridinium oxime salt now having the desired anion A₂⁻. A₂ in HA₂ may therefore include, but is not limited to, a chloride —Cl (as in HCl) or —OSO₂CH₃ (as in methane sulfonic acid CH₃SO₃H). A₂ would therefore be the corresponding chloride anion Clor the methane sulfonate anion CH₃SO₃⁻.

In the broad context of the present invention, it can now be appreciated that the first pyridinium oxime salt for ion exchange may have the following general structure

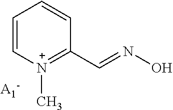

and the second pyridinium oxime salt formed herein has the general structure illustrated below. Accordingly, it can be appreciated that the second pyridinium oxime salt may include pralidoxime chloride.

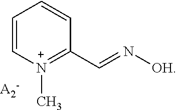

It may also be appreciated that the first pyridinium oxime salt for ion exchange may have the following general structure

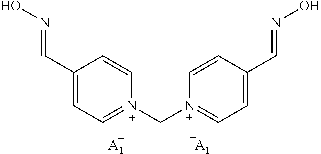

and the second pyridinium oxime salt formed herein has the general structure shown below. Accordingly it can be appreciated that the second pyridinium oxime salt may include 1,1′-methylenebis(4-((hydroxyimino)methyl)-pyridinium)dimethanesulfonate or MMB-4 DMS.

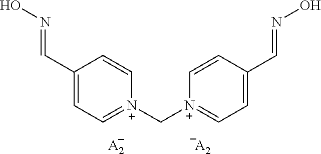

In addition, the first pyridinium oxime salt for ion exchange may have the following general structure

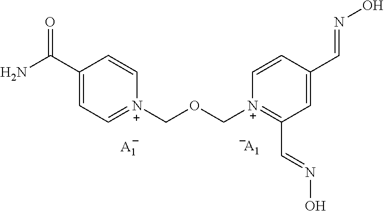

and the second pyridinium oxime salt formed herein has the general structure shown below. Accordingly, it can be appreciated that the second pyridinium oxime salt can include 1-[({2,4-bis[(E)-(hydroxyimino)methyl]-1-pyridiniumyl}methoxy)methyl]-4-carbamoylpyridinium dimethanesulfonate or HLö-7 DMS.

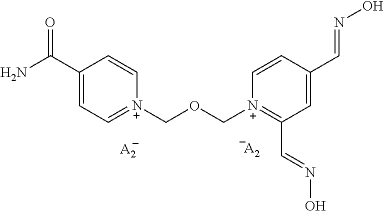

Moreover, the first pyridinium oxime salt for ion exchange may have the following general structure

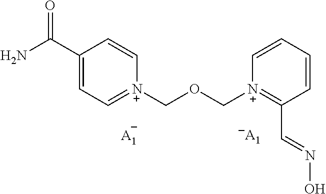

and the second pyridinium oxime salt formed herein has the structure shown below. Accordingly, it can be appreciated that the second pyridinium oxime salt includes (1-(2-(hydroxyiminomethyl)pyridinium)-3-(4-carbamoylpyridinium)-2-oxapropane)dimethanesulfonate or HI-6 DMS.

In addition, the first pyridinium oxime salt for ion exchange may have the following general structure

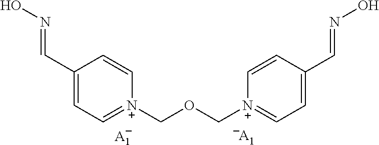

and the second pyridinium oxime salt formed herein has the structure shown below. Accordingly, it can be appreciated that the second pyridinium oxime salt includes 1,1′(oxydimethylenebis(4-formylpyridinium)dioxime dimethanesulfonate or obidoxime DMS.

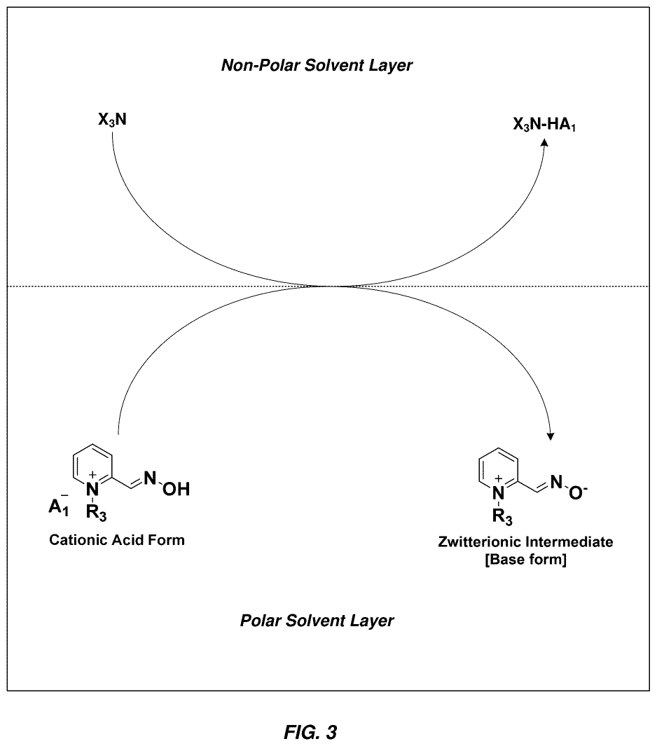

Attention is next directed to FIG. 3 which shows a particular preferred procedure for the ion-exchange of a first cationic pyridinium oxime (first pyridinium oxime salt) having an anion (A₁⁻) for replacement via the formation and use of zwitterionic intermediate. More specifically, FIG. 3 now illustrates the preferred phase separation procedure where one can rely upon a tertiary amine (X₃N) that preferably has solubility in relatively non-polar solvents, such as hydrocarbon type solvents (e.g., hexane) and is generally insoluble in relatively polar solvents such as water, dimethylformamide (DMF) or ethylene glycol. On that note, X₃N is preferably selected from trioctylamine, where X is an octyl hydrocarbon group, more preferably n-octyl, which can then be identified with the general formula N(n-Oct)₃. Trioctylamine reportedly has a solubility in water of 0.050 mg/l at 25° C.

As therefore illustrated in FIG. 3, the tertiary amine X₃N is selected such that it remains soluble in relatively non-polar solvents, such as a hydrocarbon solvent, which would preferably include hexane or toluene. The cationic form of the first pyridine oxime salt having the undesired anion A₁⁻ is soluble in the polar solvent layer, which as noted, may include water, DMF or ethylene glycol. At the phase boundary the tertiary amine (e.g., N(n-Oct)₃) will both remove HA₁ and provide a quantitative conversion of the identified pyridinium oxime to the zwitterionic intermediate (base form) which zwitterionic intermediate remains in the polar solvent layer. FIG. 3 therefore shows the situation with the first pyridinium oxime salt having the following general structure below where R₁ is a hydrogen and R₂ is the oxime group and R₃ is as noted above.

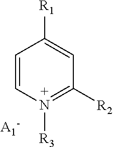

Accordingly, it can now be appreciated that the other zwitterionic intermediates that may be formed herein include any one of the following:

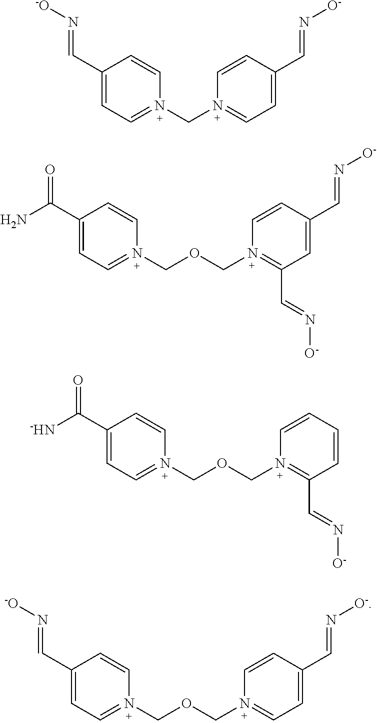

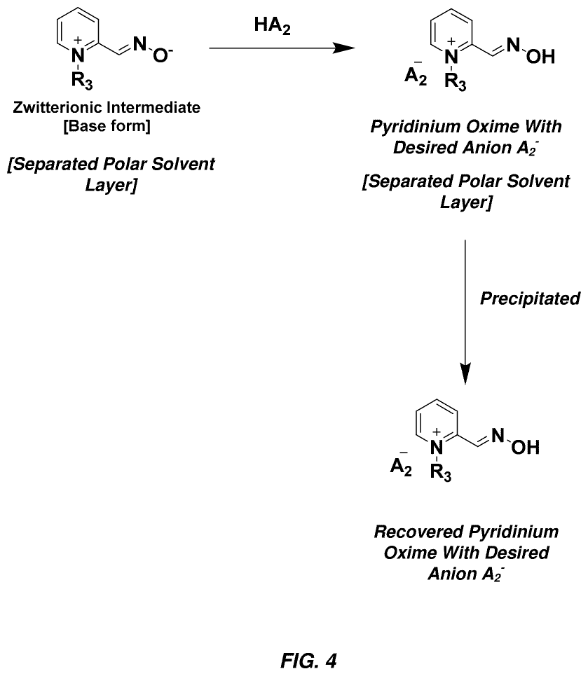

The layers in FIG. 3 are therefore conveniently separated and as next shown in FIG. 4, the zwitterionic intermediate in the now separated polar solvent layer may then be treated with acid HA₂ where A₂ will provide the desired anion to be introduced into the pyridinium oxime. Within the separated polar solvent layer the identified second pyridinium oxime salt with the desired anion A₂⁻ is now formed which can be conveniently precipitated from such medium and recovered.

As may now be appreciated, the present invention provides for ion-exchange of a first cationic pyridinium oxime (first pyridinium oxime salt) having an anion for replacement via the formation and use of zwitterionic intermediates that are preferably recovered by phase separation followed by conversion into a second cationic pyridinium oxime (second pyridinium oxime salt) having the desired anion. The process avoids the use of anion exchange resin treatment of pyridinium oxime salts. The process also is contemplated to provide a relatively more economical solution to the multi-kilogram production of desired pyridinium oxime salts for treatment of organophosphorus nerve agent poisoning.

What is claimed is:

1. A method of exchanging anionic groups in a pyridinium oxime salt comprising:
a. providing a first pyridinium oxime salt having the following structure: 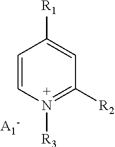
wherein A₁⁻ comprises a first anionic group, R₁ comprises a hydrogen or oxime group, R₂ comprises a hydrogen or oxime group and R₃ comprises one of:
(1) a terminal hydrocarbon group; or
(2) a hydrocarbon linking group R₄ connected to the nitrogen atom of a second pyridinium oxime as in the following: 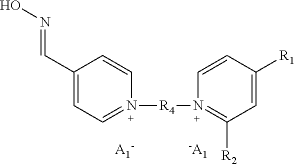
(3) a hydrocarbon ether linking group (—R₅—O—R₅—) where R₅ is a hydrocarbon connected to the nitrogen atom of a second heterocyclic ring nitrogen atom as in one of the following 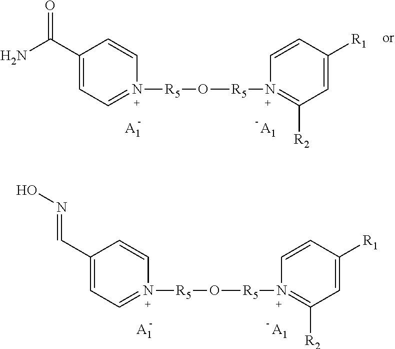
b. transforming said first pyridinium oxime salt into a zwitterionic intermediate and treating said zwitterionic intermediate with a proton acid HA₂ where H is a hydrogen and A₂ forms a second anionic group A₂⁻ thereby providing a second pyridinium oxime salt having one of the following structures: 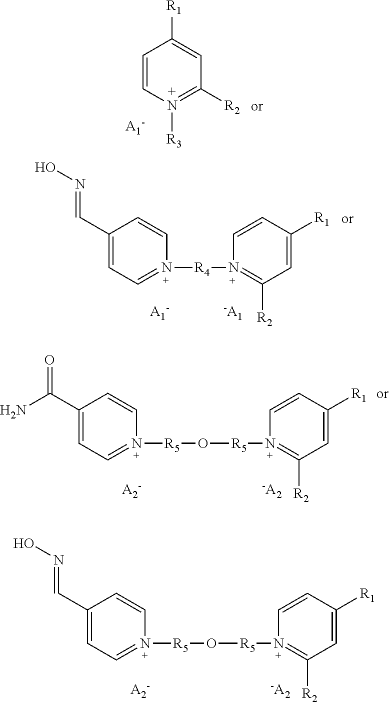
wherein A₂⁻ comprises an anionic group that is different from the first anionic group Ar.

2. The method of claim 1, wherein A₁⁻ comprises I⁻, Br⁻, CN⁻ or —OCN⁻.

3. The method of claim 1, wherein A₂⁻ comprises a chloride anion (Cl⁻) or a methanesulfonate anion (CH₃SO₃⁻).

4. The method of claim 1, wherein R₁ comprises a methyl group and said first pyridinium oxime salt has the following general structure: 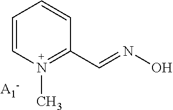
and the second pyridinium oxime salt has the following general structure:

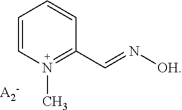

5. The method of claim 1, wherein R₁ comprises a methylene group (—CH₂—) and said first pyridinium oxime salt has the following general structure: 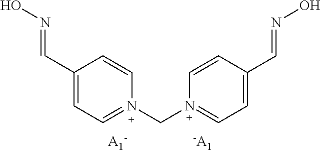
and said second pyridinium oxime salt has the following general structure:

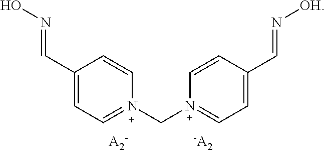

6. The method of claim 1, wherein R₁ comprises a dimethylene ether group (—CH₂—O—CH₂—) and said first pyridinium oxime salt has the following general structure: 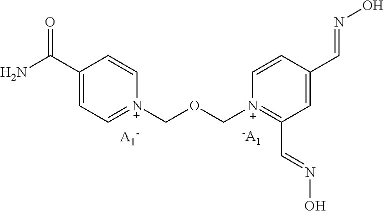
and said second pyridinium oxide salt has the following general structure:

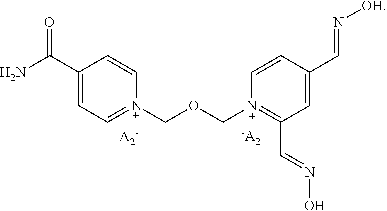

7. The method of claim 1, wherein R₁ comprises a dimethylene ether group (—CH₂—O—CH₂—) and said first pyridinium oxime salt has the following general structure: 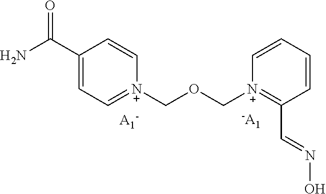
and said second pyridinium oxide salt has the following general structure:

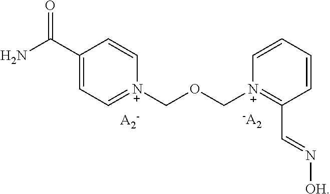

8. The method of claim 1, wherein R₁ comprises a dimethylene ether group (—CH₂—O—CH₂—) and said first pyridinium oxime salt has the following general structure: 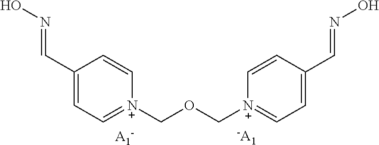
and said second pyridinium oxime salt has the following general structure:

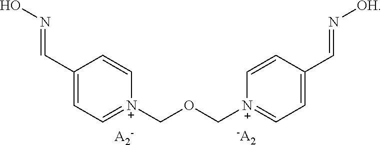

9. The method of claim 1, wherein the step of transforming said first pyridinium oxime salt into a zwitterionic compound comprises treating said first pyridinium oxime salt with a tertiary amine X₃N where X is a hydrocarbon.

10. The method of claim 1, wherein A₁⁻ comprises an iodide anion I⁻ or a bromide anion Br⁻.

11. The method of claim 1, wherein A₂⁻ comprises a chloride anion Cl— or methane sulfonate anion CH₃SO₃⁻.

12. The method of claim 1, wherein said first pyridinium oxime salt is dissolved in a polar solvent layer and said tertiary amine is dissolved in a non-polar solvent layer and said zwitterionic intermediate is formed in said polar solvent layer.

13. The method of claim 12, wherein said zwitterionic intermediate in said polar solvent layer is separated from said non-polar solvent layer and then treated with said proton acid HA₂ to provide said second pyridinium oxime salt which is then precipitated from said separated polar solvent layer.

14. A method of exchanging anionic groups in a pyridinium oxime salt comprising:
a. providing a first pyridinium oxime salt in a polar solvent layer having the following structure: 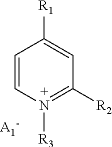
wherein A₁⁻ comprises a first anionic group, R₁ comprises a hydrogen or oxime group, R₂ comprises a hydrogen or oxime group and R₃ comprises one of:
(1) a terminal hydrocarbon group; or
(2) a hydrocarbon linking group R₄ connected to the nitrogen atom of a second pyridinium oxime as in the following: 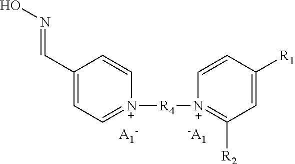
(3) a hydrocarbon ether linking group (—R₅—O—R₅—) where R₅ is a hydrocarbon connected to the nitrogen atom of a second heterocyclic ring nitrogen atom as in one of the following 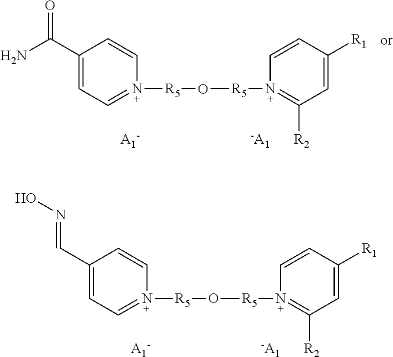
b. providing a tertiary amine X₃N in a non-polar solvent layer where X is a hydrocarbon wherein said non-polar solvent layer is in contact with said first pyridinium oxime salt in said polar solvent layer;
C. transforming said first pyridinium oxime salt in said polar solvent layer into a zwitterionic intermediate and separating said zwitterionic intermediate in said polar solvent layer from said non-polar solvent layer and treating said zwitterionic intermediate with a proton acid HA₂ where H is a hydrogen and A₂ forms a second anionic group A₂⁻ thereby providing a second pyridinium oxime salt having one of the following structures: 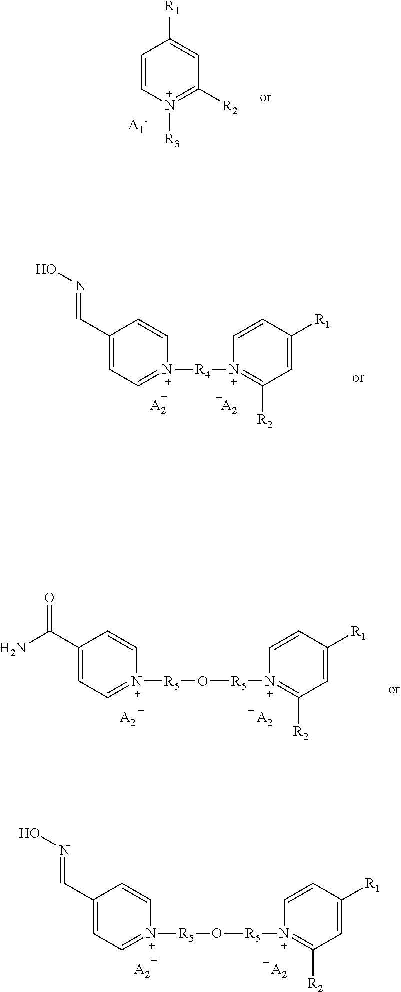
wherein A₂⁻ comprises an anionic group that is different from the first anionic group A₁.

15. The method of claim 14, wherein A₁⁻ comprises I⁻, Br⁻, CN⁻ or —OCN⁻.

16. The method of claim 14, wherein A₂⁻ comprises a chloride anion (Cl⁻) or a methanesulfonate anion (CH₃SO₃⁻).

17. The method of claim 14, wherein R₁ comprises a methyl group and said first pyridinium oxime salt has the following general structure: 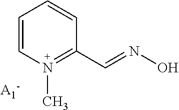
and the second pyridinium oxime salt has the following general structure:

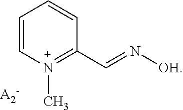

18. The method of claim 14, wherein R₁ comprises a methylene group (—CH₂—) and said first pyridinium oxime salt has the following general structure: 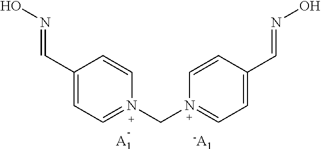
and said second pyridinium oxime salt has the following general structure:

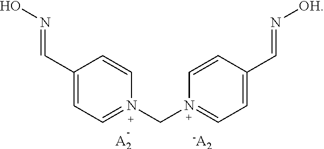

19. The method of claim 14, wherein R₁ comprises a dimethylene ether group (—CH₂—O—CH₂—) and said first pyridinium oxime salt has the following general structure: 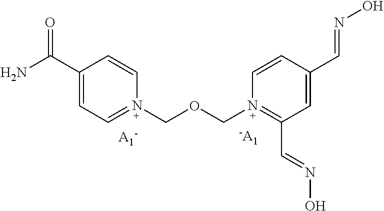
and said second pyridinium oxide salt has the following general structure:

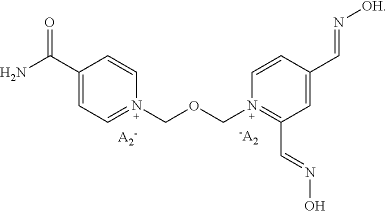

20. The method of claim 14, wherein R₁ comprises a dimethylene ether group (—CH₂—O—CH₂—) and said first pyridinium oxime salt has the following general structure: 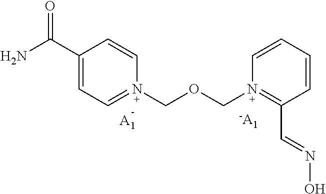
and said second pyridinium oxide salt has the following general structure:

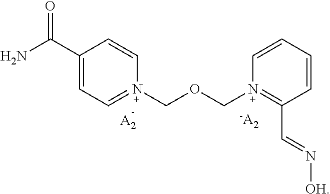

21. The method of claim 14, wherein R₁ comprises a dimethylene ether group (—CH₂—O—CH₂—) and said first pyridinium oxime salt has the following general structure: 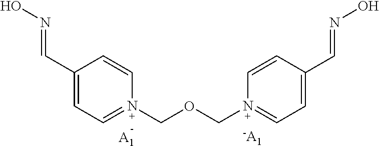
and said second pyridinium oxime salt has the following general structure:

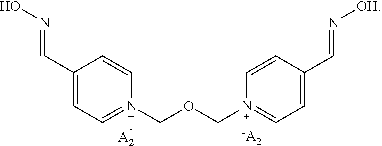

22. A method of exchanging anionic groups in a pyridinium oxime salt comprising:
a. providing a first pyridinium oxime salt in a polar solvent layer having the following structure: 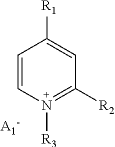
wherein A₁⁻ comprises a first anionic group, R₁ comprises a hydrogen or oxime group, R₂ comprises a hydrogen or oxime group and R₃ comprises one of:
(1) a terminal hydrocarbon group; or
(2) a hydrocarbon linking group R₄ connected to the nitrogen atom of a second pyridinium oxime as in the following: 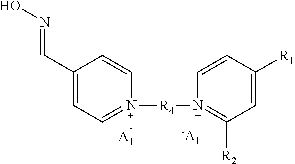
(3) a hydrocarbon ether linking group (—R₅—O—R₅—) where R₅ is a hydrocarbon connected to the nitrogen atom of a second heterocyclic ring nitrogen atom as in one of the following 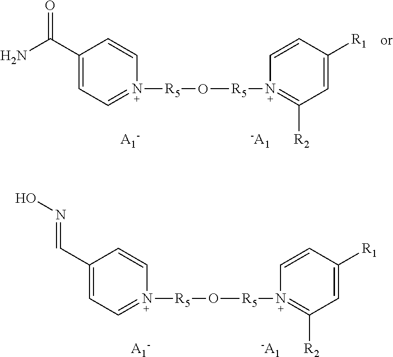
b. providing a tertiary amine X₃N in a non-polar solvent layer where X is an octyl hydrocarbon and said non-polar solvent layer is in contact with said first pyridinium oxime salt in said polar solvent layer;
c. transforming said first pyridinium oxime salt in said polar solvent layer into a zwitterionic intermediate and separating said zwitterionic intermediate in said polar solvent layer from said non-polar solvent layer and treating said zwitterionic intermediate with a proton acid HA₂ where H is a hydrogen and A₂ forms a second anionic group A₂⁻ thereby providing a second pyridinium oxime salt having one of the following structures: 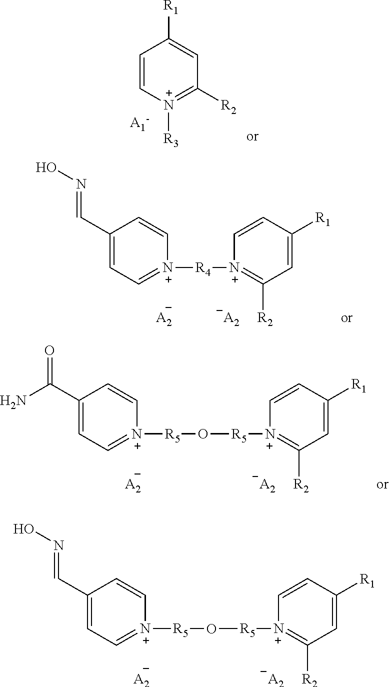
wherein A₂⁻ comprises an anionic group that is different from the first anionic group A₁⁻.
